(19) United States
(12) Patent Application Publication
(10) Pub. No.: US 2009/0287140 A1
(43) Pub. Date: Nov. 19, 2009

(54) ELECTRICAL STIMULATION AND INFUSION INTRODUCER ASSEMBLY

(76) Inventor: William J. Rittman, III, Lynnfield, MA (US)

Correspondence Address: SHERIDAN ROSS PC, 1560 BROADWAY, SUITE 1200, DENVER, CO, 80202, US

(21) Appl. No.: 12/122,266

(22) Filed: May 16, 2008

(51) Int. Cl. A61M 5/158; A61B 18/14

(52) U.S. Cl. 604/21

(57) ABSTRACT

An electrical stimulation and infusion introducer assembly is provided for connection to an electrical electrode/electrode. The electrode extends through the lumen of an introducer needle of the introducer assembly. The introducer assembly includes a side port for introduction of liquids through the introducer needle without having to remove the electrode. The side port communicates with the central passageway of the introducer assembly, and the central passageway in turn communicates with the lumen of the needle.

### FIELD OF THE INVENTION

The present invention relates generally to processes, systems, and devices for the treatment of pain, neurological disorders, and related to neural dysfunction. More specifically, the present invention relates to a device to produce therapeutic lesions or tissue alterations by a stimulation probe having an electrode element that delivers high-frequency energy to targeted tissue, and further more specifically to an electrical stimulation and infusion introducer assembly that can deliver an anesthetic to the targeted issue while holding the electrode element during a procedure without having to remove the stimulation probe from the introducer assembly.

### BACKGROUND OF THE INVENTION

It is well known to use radio-frequency generators and electrodes to apply radio-frequency energy in order to treat pain and/or to modify neural tissue for other treatment purposes. Heating of the tissue is caused by RF resistance power dissipation. The amount of heat applied is monitored by a temperature-sensing element. In addition to temperature, other parameters that may be measured by the RF generator and accompanying systems include impedance, voltage, current, and power. Various parameters may be set by the user in the delivering of energy to include the time of delivery, desired electrode temperature, stimulation frequencies and durations, and the level of stimulation output.

Heating tissue from between about 60° to 95° C. is common, it being known that a great portion of tissue may die at temperatures at between 45° to 50° C. The heating of the tissue in this manner often causes significant pain to the patient thereby necessitating the use of a local anesthetic.

In one example of a treatment protocol, a patient may complain of back pain or some other pain of neuropathic or nocioceptive origin. The practitioner will typically first perform diagnostic blocks with local anesthetic by injecting the anesthetic into the areas that are suspected of generating the pain. If the patient receives temporary relief from these injections, then the practitioner may conclude that the pain generators are in the general location where the injections were made. However, in order to modify or destroy the neural tissue responsible for the pain generation, the practitioner must then more specifically identify the exact location where pain is generated. An RF electrode is typically emplaced into the patient through an introducer needle, the electrode being disposed coaxially inside the introducer needle. The practitioner will take advantage of an imaging technology such as fluoroscopy in order to precisely position the introducer needle. The electrode is insulated, with a small non-insulated electrically active tip placed at the targeted location within the patient. Once the introducer needle is placed at the targeted location, it is withdrawn thus exposing the electrode. Alternatively, the electrode may have multiple active areas separated by non-conductive areas that provide capability to generate larger lesions in a single procedure. First, stimulation pulses are delivered by applying a voltage to the electrode placed at the suspected pain generating area. This voltage (for example, about 0.5 volts) is applied at a sensory frequency (typically 50 Hz) that should result in a very low threshold response from the patient. Assuming the correct response is received from the targeted sensory nerves, another stimulation test is conducted at a muscle motor frequency (for example, 2 Hz), and at an increased voltage (approximately 2 volts). In this test, the practitioner looks for no motor response in the patient's extremities, as otherwise this would indicate that the electrode was too close to the motor nerves. Treatment to the motor nerves could lead to paralysis.

Upon successful completion of these initial tests, the targeted neural tissue is destroyed or modified by delivering a high-frequency energy through the electrode. A ground or reference plate is placed on the patient's thigh to provide a ground or return path for the high-frequency energy provided by the electrode. The high-frequency energy is typically delivered for one or more minutes while maintaining an electrode tip temperature between about 70° to 90° C. Alternatively, high-frequency energy may be delivered for one or more minutes, but in a pulsed mode where the high-frequency energy is on for short periods of time and off for longer periods of time.

Prior to conducting the actual tissue ablation or modification procedure, the electrode is removed from within the introducer needle so a local anesthetic can be delivered to the targeted area through a syringe attached to the base of the introducer needle. The syringe is then disengaged from the introducer needle and the electrode is replaced for the heating phase of the procedure. The most common connection used between the syringe and the base of the introducer needle is a luer lock or luer connection. The luer connection is well known in the industry, and essentially consists of a threaded fitting placed between the end of the syringe and the end of the introducer needle assembly. During the introduction of the anesthetic, it is quite possible that the introducer needle may slight shift. Thus, upon reinserting the electrode through the introducer needle, the electrode will not be returned to its previous position within the patient established during testing. Particularly when the introducer needle is withdrawn a certain length to expose the tip of the electrode, it is quite difficult to push the introducer needle back into the patient to its exact previous testing location and thus, the electrode will inherently not return to its original testing location.

Therefore, there is a need for an introducer needle assembly that can be used to deliver anesthesia to the targeted area but does not require the electrode to be removed from within the introducer needle. There is also a need to provide an introducer needle assembly that can maintain the required positive locking relationship with the electrode during the procedure.

Therefore, it is one object of the invention to provide an electrical stimulation introducer assembly that provides positive locking between the introducer needle and an electrode received through the introducer needle. It is yet another object of the present invention to provide the capability to infuse liquids such as an anesthetic or other medications directly through the introducer needle without having to remove the electrode, thereby assuring that the electrode does not drift from its original location established during sensory and motor stimulation. It is yet another object of the present invention to provide an introducer needle assembly that is simple in construction, yet is reliable and adapted to receive existing electrodes.

### SUMMARY OF THE INVENTION

In accordance with the present invention, an electrical stimulation and infusion introducer assembly is provided having a side port that allows introduction of a liquid such as an anesthetic through the introducer assembly without having to remove a stimulation probe/electrode inserted through the introducer needle of the assembly. The assembly also includes exterior locking elements that enable the electrode to be secured to the introducer needle without having to twist or rotate either the electrode or the assembly, thereby further assuring proper placement of the introducer needle and electrode.

In a preferred embodiment of the present invention, the introducer assembly includes a hub or body with one end connected to an introducer needle. The opposite end of the body is open to receive the electrode. More specifically, the body includes a central passageway and the open end is adapted to frictionally and sealingly engage a base portion of the electrode. The probe or electrode extends through the central passageway and through the central lumen of the introducer needle. A side port is formed on the body of the introducer assembly, and the side port communicates with the central passageway. An external tube may connect to the side port, and the tube can interconnect the side port to a syringe that supplies a liquid. Since the electrode does not completely fill the lumen of the introducer needle, the liquid travels through the introducer needle to the targeted area of tissue while the electrode remains in place within the introducer needle. The base of the electrode is firmly seated within the hub of the assembly by a seal within the central passageway. Thus, fluid is prevented from escaping the central passageway even when under considerable pressure, such as created by injection of fluid through a syringe. A practitioner can therefore reliably deliver fluid such as an anesthetic directly through the introducer assembly without having to remove the electrode.

The body of the introducer assembly also incorporates an exterior locking mechanism that prevents inadvertent disconnection of the electrode from the introducer assembly. The locking mechanism includes a pair of locking tabs that engage an external peripheral groove of the electrode base. Proper seating of the electrode within the introducer assembly is signaled by an audible click caused by the resilient locking movement of the locking tabs into the peripheral groove. This locking movement also provides a tactile indication of proper seating.

In one aspect of the present invention, it may be considered an electrical stimulation and infusion introducer assembly. In another aspect of the present invention, it may be considered the combination of the introducer assembly and an electrical stimulation probe/electrode. In yet another aspect of the present invention, it may be considered various methods to include a method of connecting an electrode to an introducer assembly, and a method of treatment using the introducer assembly wherein delivery of liquids through the introducer needle can be achieved without removal of the electrode within the needle.

These features of the invention along with others will become apparent from a review of the following detailed description taken in conjunction with the corresponding Figures.

### BRIEF DESCRIPTION OF THE DRAWINGS

FIG. 1 is a fragmentary perspective view of the electrical stimulation and infusion introducer assembly of the present invention;

FIG. 2 is a cross-section of FIG. 1;

FIG. 3 is another cross-section of FIG. 1;

FIG. 4 is an end view of the introducer assembly;

FIG. 5 is a fragmentary perspective view of one example of an electrode usable with the introducer assembly of the present invention;

FIG. 6 is a cross-section of the electrode of FIG. 5 connected to the introducer assembly; and

FIG. 7 is another cross-section illustrating the connection between the electrode and the introducer assembly.

### DETAILED DESCRIPTION

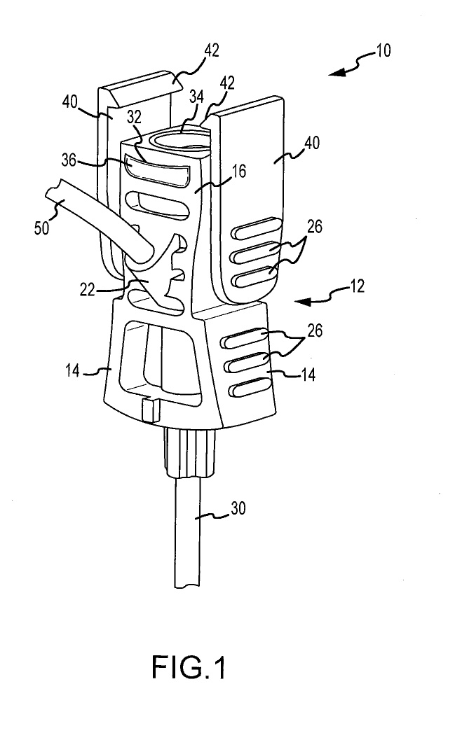

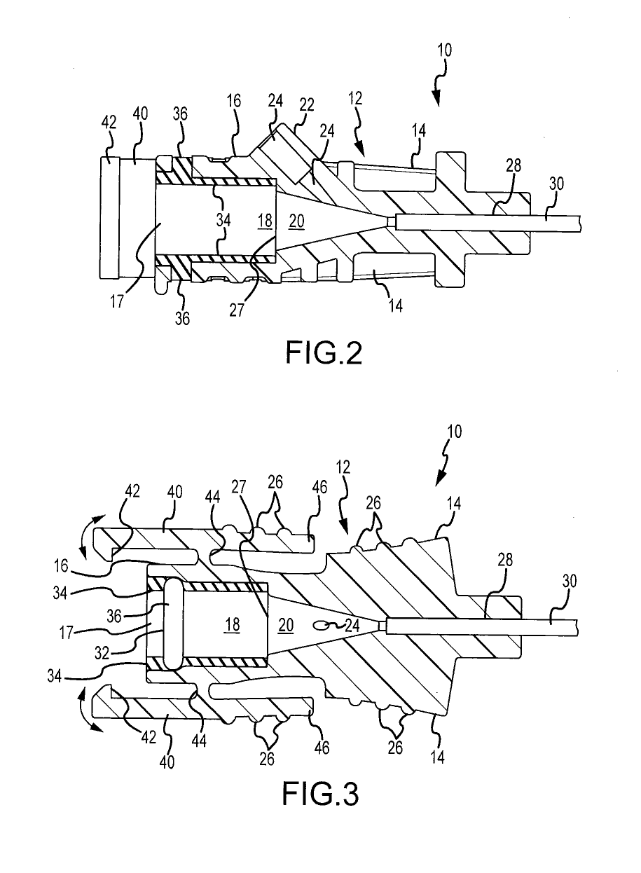

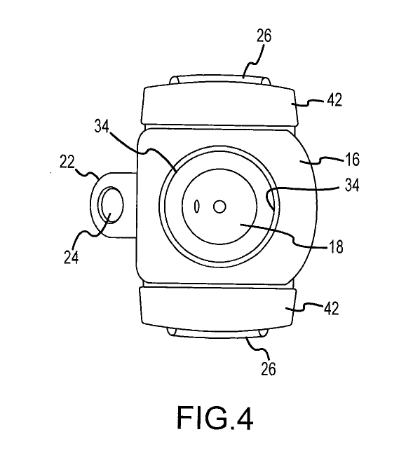

Referring to FIGS. 1-4, the electrical stimulation and infusion introducer assembly 10 is shown. The introducer assembly 10 includes a hub or body 12. The hub/body 12 may be further defined as having a needle receiving portion 14 and an extension 16 that receives an electrode. Portion 14 includes a needle port 28 that receives an introducer needle 30. The body 12 further includes a central passageway 18 that extends between the open end 17 and the needle port 28. The central passageway includes a substantially cylindrical portion communicating with the open end 17, and a narrowing portion 20 that communicates with the needle port 28. A side port 22 is formed on the hub extension 16. The side port 22 includes a side port passageway 24 that communicates with the narrowing portion of the central passageway 18. As shown in FIG. 2, the side port passageway 24 includes an upper cylindrical portion that may be sized to frictionally engage a length of tubing 50. A gasket 34 is received in the cylindrical portion of the central passageway 18. The gasket 34 may include pair of gasket extensions 36 that is received in respective opposite side openings 32 formed in the extension 16. The gasket extensions 36 provide additional anchoring to ensure proper positioning of the gasket 34 within the central passageway.

As best seen in FIG. 3, a pair of opposing locking tabs 40 attach to the body 12 by respective bridge sections 44. The locking tabs 40 extend beyond the open end 17 of the body 12 and have hooks 42 formed on the respective ends of the tabs 40. The opposite ends 46 of the locking tabs extend substantially parallel with the outer surface of the extension 16. A plurality of gripping elements 26 may be formed on the ends 46, and these elements as best seen in FIG. 1 are simply small groups of protrusions. As also shown in FIG. 1, the base 14 may include a plurality of gripping elements 26.

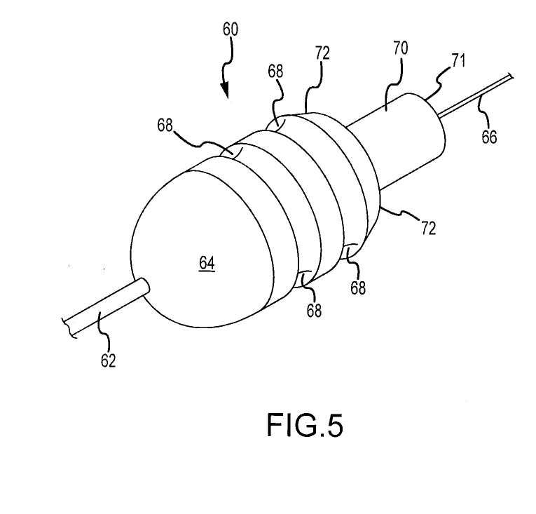

Referring to FIG. 5, one example is provided of an electrical stimulation probe or electrode 60. The electrode 60 includes a power/signal cord 62, a base 64, and an electrode element 66 that protrudes from a cylindrical portion 70 of the base 64. The outer surface of the base 64 may include a plurality of spaced circumferential grooves 68.

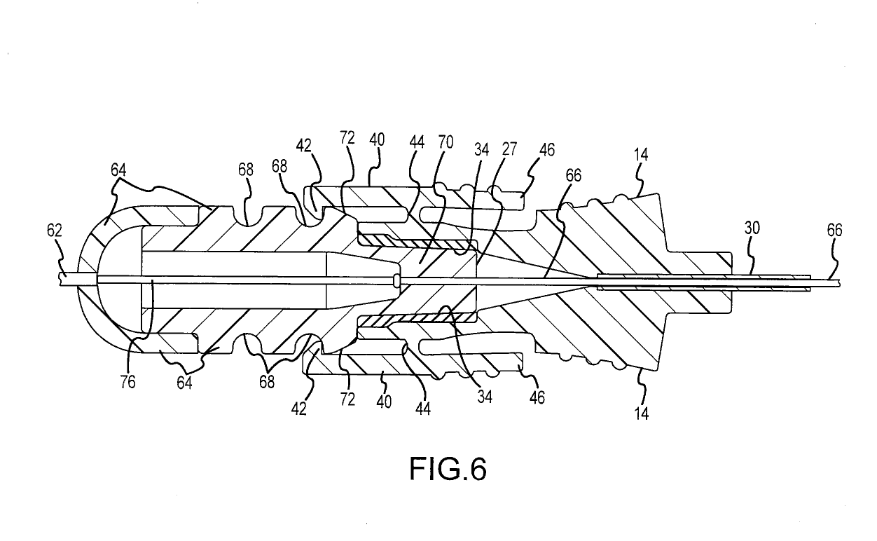

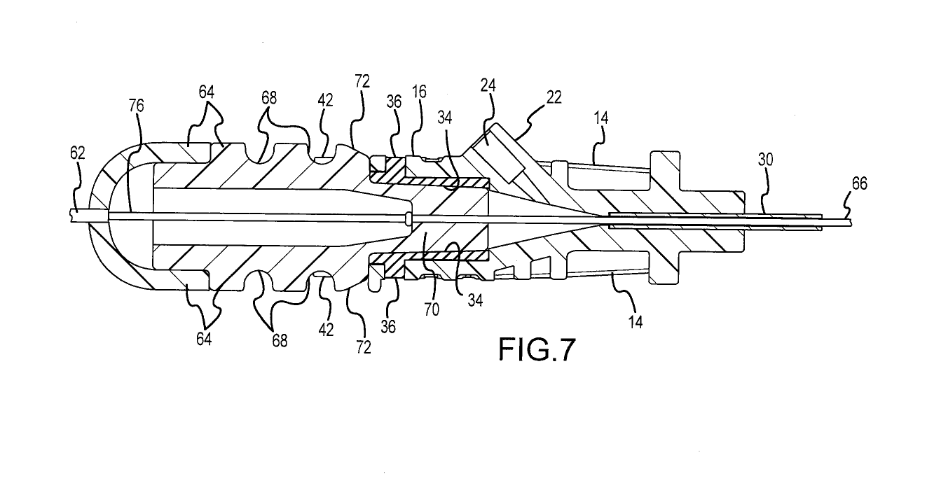

Referring to FIGS. 6 and 7, the electrode 60 is shown connected with the introducer assembly 10. The interior of electrode base 64 interconnects the electrode element 66 with the power/signal line 62 and may include an electrical connecting cable/wire bundle 76. Additional elements may be incorporated within the base 64 to include circuitry, sensors, or other functional elements. The electrode element 66 is inserted through the central lumen of the introducer needle 30. The cylindrical portion 70 of the electrode base 64 is seated within the central passageway 18 and frictionally engages the gasket 34. Thus, a sealed connection is created between the cylindrical portion 70 and the gasket 34.

In order to connect the electrode 60 to the assembly 10, a user simply inserts the electrode element 66 into the central passageway 18 and through the lumen of the introducer needle 30. The insertion is complete when the leading end 71 of the cylindrical portion 70 reaches the interior end wall 27 of the cylindrical portion of the central passageway. As the electrode 60 is inserted within the introducer assembly and prior to full insertion, the hooks 42 contact the leading curved edge 72 of the electrode base 64. When the electrode is fully inserted and seated, the hooks 42 will snap in place within groove 68. This snapping action has an audible clicking sound, as well as a tactile indication of attachment. The electrode 60 can be removed by depressing radially inward on the activation ends 46 of the locking tabs, thus rotating the hooks 42 outward away from within the groove 68, and then pulling the electrode 60 away from the introducer assembly. The bridges 44 provide some resiliency in maintaining the hooks 42 in a normally locked position within the groove 68.

During a procedure, the electrode remains inserted within the introducer assembly during sensory stimulation and motor stimulation. An anesthetic or any other desired liquid could be introduced through the side port 22 and directly to the targeted tissue without having to remove the electrode. As mentioned, the electrode 66 is not large enough to completely fill the central lumen within the introducer needle 30 therefore allowing the liquid to travel through the introducer needle and directly to the targeted tissue where the active tip of the electrode element 66 is located. Thus, in accordance with the present invention, the electrode remaining in place after stimulation and motor testing ensures that the electrode does not drift within the patient, and such drifting or shifting could otherwise jeopardize the procedure.

There are a number of advantages to the present invention. An introducer assembly is provided that not only has the capability of accepting a common electrical stimulation device, but also has the capability to engage the stimulation device with self-locking tabs that automatically engage the forward end of the electrode base. The side port of the introducer assembly allows introduction of liquids through the introducer needle without the requirement to remove or reposition the electrode element. Another advantage is that locking of the introducer assembly to the electrode does not require any rotation or twisting of either the electrode or the introducer assembly; rather, the external and parallel arrangement of the locking tabs enable connection by simply inserting the electrode.

While the present invention has been disclosed with respect to one or more embodiments, it shall be understood that various other changes and modifications may be made to the present invention in accordance with the scope of the claims appended hereto.

What is claimed is:

1. An electrical stimulation and infusion introducer assembly comprising:
a body having a first open end, a second end, and a central passageway extending through the body along a longitudinal axis thereof;
a side port formed on said body and extending transversely to said central passageway and communicating with said central passageway;
an introducer needle secured to said second end, said introducer needle having a lumen communicating with said central passageway;
a gasket placed in said central passageway adjacent said first end of said body; and
a pair of locking tabs formed on an outer surface of said body, said locking tabs being moveable between locked and unlocked positions.

2. An assembly, as claimed in claim 1, wherein:
said central passageway has a cylindrical shape at said first end of said body.

3. An assembly, as claimed in claim 1, wherein:
said locking tabs extend longitudinally along and spaced from said body, said tabs having first ends extending beyond first end of said body, and each tab having a hook formed thereon at said first end of said tab.

4. An assembly, as claimed in claim 1, wherein:
each tab further include a bridge to interconnect the tab to said body, said bridge being resilient such that said tab is urged to a normally locked position directed radially inward.

5. An assembly, as claimed in claim 1, wherein:
said gasket has securing extensions that extend through said body for maintaining said gasket within said central passageway.

6. An assembly, as claimed in claim 1, further including:
a plurality of gripping elements formed on said body and said locking tabs.

7. In combination, an electrical stimulation and infusion introducer assembly and stimulation probe, said combination comprising:
(a) an introducer assembly including:
a body having a first open end and a second end, said body having a central passageway extending therethrough along a longitudinal axis thereof;
a side port formed on said body and extending transversely to said central passageway and communicating with said central passageway;
an introducer needle secured to said second end, said introducer needle having a lumen communicating with said central passageway;
a gasket placed in said central passageway adjacent said first end of said body;
a pair of locking tabs formed on an outer surface of said body, said locking tabs being moveable between locked and unlocked positions; and
(b) a stimulation probe having: a base and an electrode element extending from said base, said base having a leading portion thereof received in said central passageway and said leading portion making sealing contact with said gasket, and wherein said electrode element extends through said central lumen of said introducer needle.

8. The combination, as claimed in claim 7, wherein:
said stimulation probe further includes at least one outer circumferential groove, and wherein said hooks of said locking tabs are received in said groove when said stimulation probe is fully inserted within said introducer assembly.

9. The combination, as claimed in claim 7, wherein:
said central passageway has a cylindrical shape adjacent said first end of said body.

10. The combination, as claimed in claim 7, wherein:
said locking tabs extend longitudinally along and spaced from said body, said tabs having first ends extending beyond first end of said body, and each tab having a hook formed thereon at said first end of said tab.

11. The combination, as claimed in claim 7, wherein:
each tab further includes a bridge to interconnect the tab to said body, said bridge being resilient such that said tab is urged to a normally locked position directed radially inward.

12. The combination, as claimed in claim 7, wherein:
said gasket has securing extensions that extend through said body for maintaining said gasket within said central passageway.

13. The combination, as claimed in claim 7, wherein:
a plurality of gripping elements formed on said body and said locking tabs.

14. A method of attaching a stimulation probe to an introducer assembly, said method comprising the steps of:
providing an introducer assembly comprising:
a body having a first open end and a second end, said body having a central passageway extending therethrough along a longitudinal axis thereof;
a side port formed on said body and extending transversely to said central passageway and communicating with said central passageway;
an introducer needle secured to said second end, said introducer needle having a lumen communicating with said central passageway;
a gasket placed in said central passageway adjacent said first end of said body; a pair of locking tabs formed on an outer surface of said body, said locking tabs being moveable between locked and unlocked positions;
providing a stimulation probe comprising a stimulation probe having a base and an electrode element extending from said base;
inserting the electrode element of the stimulation probe through said central passageway and into said lumen of the introducer needle;
further inserting the stimulation probe so a leading portion of the base is received in the central passageway and makes sealing contact with the gasket;
contacting the hooks of the tabs with the leading portion of the stimulation probe; and
snapping the hooks of the tabs in a circumferential groove of the base of the stimulation probe wherein said tabs are resiliently held within said circumferential groove.

15. A method of connecting a stimulation probe to an introducer assembly in order to provide stimulation of targeted tissue within a patient, said method comprising the steps of:
(a) providing an introducer assembly comprising:
a body having a first open end and a second end, said body having a central passageway extending therethrough along a longitudinal axis thereof;
a side port formed on said body and extending transversely to said central passageway and communicating with said central passageway;
an introducer needle secured to said second end, said introducer needle having a lumen communicating with said central passageway;
a gasket placed in said central passageway adjacent said first end of said body;
a pair of locking tabs formed on an outer surface of said body, said locking tabs being moveable between locked and unlocked positions;
(b) providing a stimulation lead comprising: a base and an electrode element extending from said base, said base having a leading portion thereof received in said central passageway and said leading portion making sealing contact with said gasket, and wherein said electrode element extends through said central lumen of said introducer needle;
(c) placing the introducer needle into the body of a patient at a targeted area for stimulation by said electrode element; and
(d) introducing a desired liquid through the side port of the introducer assembly to the targeted area within the patient while the stimulation probe remains inserted through said introducer assembly.
